(19) United States
(12) Patent Application Publication
(10) Pub. No.: US 2007/0292369 A1
(43) Pub. Date: Dec. 20, 2007

(54) METHOD FOR PREVENTING AND REDUCING TARTAR BUILDUP

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/749,104

(22) Filed: May 15, 2007

Related U.S. Application Data
(60) Provisional application No. 60/800,918, filed on May 15, 2006.
(60) Provisional application No. 60/800,638, filed on May 15, 2006.
(60) Provisional application No. 60/800,631, filed on May 15, 2006.

(51) Int. Cl. A61K 8/00; A61K 8/18; A61K 8/46; A61K 8/97; A61P 43/00

(52) U.S. Cl. 424/53; 424/49; 424/56; 424/57; 424/58

(57) ABSTRACT

A method of reducing tartar on a surface includes providing a composition including at least one active ingredient for reducing tartar, which composition may be provided in a container. The composition is configured to maintain the active ingredient in contact with the surface for an extended time. The composition may have a viscosity greater than about 20,000 cp and/or may include at least one thickener and/or additive. The method also includes applying the composition to the surface and allowing the composition to remain in contact with the surface for an extended time.

### CROSS REFERENCE TO RELATED APPLICATIONS

This application claims the benefit of U.S. Patent Application Ser. No. 60/800,918, entitled COMPOSITION TO PRECENT AND REDUCE TARTAR BUIDUP AND METHOD OF USING SAME, filed May 15, 2006; U.S. Patent Application Ser. No. 60/800,638, entitled METHOD AND SYSTEM FOR FACILITATING ORAL HEALTH, filed May 15, 2006; and U.S. Patent Application Ser. No. 60/800,631, entitled METHOD AND SYSTEM FOR FACILITATING AND MAINTAINING ORAL HEALTH THROUGH PRESCRIBED APPLICATIONS OF ORAL COMPOSITIONS, filed May 15, 2006.

### FIELD OF INVENTION

The present invention generally relates to methods for promoting and maintaining oral health and hygiene. More particularly, the invention relates to methods of using compositions to prevent and reduce tartar buildup, such as by removing existing tartar deposits and/or reducing the accumulation of additional tartar.

### BACKGROUND OF THE INVENTION

Unfortunately, poor oral health affects millions of people every year. Poor oral health may result in symptoms ranging from bad breath, tooth decay, and tooth coloration, to more serious health problems, such as gum disease, tooth loss, and even general health problems, such as heart disease, stroke, poorly controlled diabetes and preterm labor. Poor oral health generally begins with the accumulation of dental deposits, such as plaque and/or tartar.

The presence of dental plaque, or simply plaque, in an oral cavity can lead to such oral and general health problems. Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing and flossing the affected areas. If not removed, however, the presence of plaque can give rise to tartar formation, tooth decay, gingivitis, periodontitis, and other health problems.

Tartar, also referred to as calculus, is a hard, calcified plaque material that exhibits a yellowish or brownish color. Tartar forms as a result of minerals (e.g., those present in saliva and gum pockets) reacting with plaque material to form a rough calculus. Calculus generally arises from the nucleation of calcium phosphate, often in areas where the large salivary gland ducts secrete their saliva. Calculus can form on surfaces not covered by the oral mucosa (supragingival) or on surfaces located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth can, in turn, lead to tooth decay, gum disease, tooth loss, as well as systemic health problems.

In addition to the health concerns, tartar is a cosmetic problem due to its discoloration of teeth. Namely, teeth can become yellowish or brownish in color. Moreover, because the surface of tartar is rough and porous, the tartar absorbs colors from other sources (e.g., coffee, tea, tobacco, smoke, red wine and the like), and thus the presence of tartar exacerbates cosmetic tooth coloration typically associated with such other sources.

Typical methods of preventing tartar buildup include brushing with a tartar control toothpaste. Although such toothpastes, if used regularly, may prevent additional buildup of tartar, the toothpastes are not thought to be effective at removing existing tartar from tooth and device surfaces.

Methods of removing existing tartar typically include scaling or root planing, both of which are performed by dentists or hygienists with the aid of specialized tools. Although these techniques work well, they are relatively expensive and time consuming. Furthermore, various methods for inhibiting tartar may cause damage to tooth enamel and/or to dental devices.

Accordingly, improved systems and techniques for reducing dental deposits are desired. Such improved systems and methods may reduce a user's current dental deposit level by removing existing tartar and/or plaque deposits (referred to herein as removal), by reducing the rate at which new plaque and/or tartar are deposited on the surfaces (referred to herein as prevention), or by a combination of removal and prevention.

### SUMMARY OF THE INVENTION

In accordance with various aspects of the present invention, a method for preventing and reducing tartar buildup is provided. The various exemplary methods described herein may employ various exemplary oral health care kits, systems, compositions, and devices to provide a relatively inexpensive and safe treatment for reducing, preventing, or mitigating tartar buildup. The various exemplary methods are relatively easy to use or perform, do not require a visit to a dentist office, and do not damage the surface of enamel.

In accordance with various exemplary embodiments of the invention, a method for reducing, preventing, or mitigating tartar buildup includes providing and using a system including a composition in a container. The composition is configured to prevent or reduce tartar buildup. The container is configured for expulsion and application of the composition to a portion of an oral cavity, such as gums, teeth, gingival surface, interfaces thereof, and/or any dental device.

In accordance with various embodiments of the invention, a container is configured for application of the composition to an oral cavity. In accordance with particular examples, the container includes an expulsion or vessel portion configured to store and facilitate expulsion or other like transfer of the composition and an applicator portion configured to receive the composition and to facilitate delivery of the composition to a surface of the oral cavity. In accordance with various exemplary embodiments, the applicator portion includes an angled spout to facilitate targeted delivery of the composition—e.g., to a gum/tooth interface. In accordance with further aspects, a container is configured with an access/closure portion to maintain the composition within the container. In accordance with additional aspects of this embodiment, the container encapsulates multiple doses of the composition. Alternatively, a system includes a container for encasing a single dose.

In accordance with the method of the present disclosure, a suitable method includes providing a system including a viscous composition and a container configured to dispense the viscous composition. The viscous composition includes at least one active ingredient to promote and/or maintain oral health. In accordance with various aspects of this embodiment, the viscous composition is designed to maintain the active ingredient(s) in contact with a surface for an extended period of time. Exemplary compositions have a viscosity greater than about 20,000 centipoise (cp), preferably greater than about 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp. Suitable active ingredients include cetylpyridinium chloride (CPC), zinc salts, other antimicrobial agents, such as those described herein and other ingredients to improve oral health, such as by preventing, reducing, or mitigating tartar.

In accordance with another embodiment of the invention, a method includes providing a system including a container and a composition including a plurality of active ingredients to improve oral health. In accordance with various aspects of this embodiment of the invention, the composition includes a carrier having a thickening agent and is configured to maintain the plurality of active ingredients in contact with a surface for an extended period of time. Exemplary thickening agents suitable for use in the composition include hydroxyethylcellulose and other pharmaceutically acceptable thickeners. Exemplary active ingredients include CPC, zinc salts, other antimicrobial agents, and similar agents known to reduce or prevent buildup of tartar, plaque gingivitis, gum bleeding, periodontitis, or other disease and/or the effects of the same.

In accordance with further exemplary embodiments of the invention, a method of reducing tartar on a surface includes providing a composition that includes at least one active ingredient for reducing tartar and has a viscosity greater than about 20,000 cp. The method includes applying the composition onto a surface to prevent tartar buildup, mitigate buildup, or reduce existing buildup.

In accordance with further exemplary embodiments, a method of reducing tartar on a surface includes providing a composition having at least one active ingredient, at least one thickener ingredient, and at least one additive ingredient in a carrier. In some implementations, the at least one active ingredient, the at least one thickener ingredient, and the at least one additive ingredient are present in respective concentrations determined at least in part by the concentrations of the other ingredients. For example, additional thickener ingredients may be added when the additive ingredients are known to cause greater or lesser salivary response than average. The at least one active ingredient, the at least one thickener ingredient, and the at least one additive ingredient may be present in concentrations adapted to provide a composition having an extended surface contact time. The method further allowing the composition to remain in contact with the surface for an extended time.

In accordance with yet further exemplary embodiments within the scope of the present invention, a method includes providing a composition including a plurality of active ingredients. An exemplary composition includes cetylpyridinium chloride, a zinc salt, and a carrier.

In accordance with yet further embodiments of the invention, a method includes using a kit including a plurality of systems to prevent or reduce tartar buildup.

In accordance with various embodiments of the invention, a method includes cleaning a user's dental surface and applying the composition to the dental surface. The cleansing and the application may occur prior to a user's extended period of rest or sleep, such as after the user has finished eating and drinking before going to sleep. By applying the composition before rest, the composition is allowed to remain in contact with a dental surface for an extended time.

### BRIEF DESCRIPTION OF THE DRAWINGS

The exemplary embodiments of the present invention will be described in connection with the appended drawing figures in which like numerals denote like elements and:

FIG. 1 illustrates an exemplary system for reducing or preventing tartar buildup in accordance with an exemplary embodiment of the invention;

FIG. 2 illustrates another exemplary system for expulsion and application of a composition to an oral cavity in accordance with an exemplary embodiment of the invention;

FIG. 3 illustrates yet another system for use in accordance with various embodiments of the invention;

FIGS. 4A-4C illustrate, respectively, a left view, a front view, and a right view of an exemplary container system for use in accordance with an exemplary embodiment of the invention;

FIG. 5 illustrates a kit including a plurality of systems for use, in accordance with yet another embodiment of the invention; and

FIG. 6 illustrates a block diagram of an exemplary method for facilitating oral health care in accordance with an exemplary embodiment of the invention.

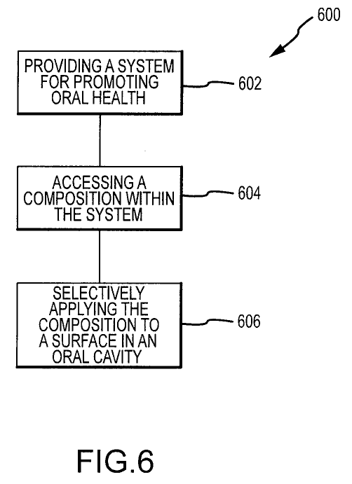

Elements in the figures are illustrated for simplicity and clarity and have not necessarily been drawn to scale. The dimensions of some of the elements in the figures may be exaggerated relative to other elements to help to improve understanding of embodiments of the present invention.

### DETAILED DESCRIPTION

The present invention provides methods of using an oral care composition, system, and/or kit to remove, prevent, or mitigate tartar buildup. The methods of the invention can be used to reduce/prevent tartar buildup on surfaces of various animals, and are particularly well suited for the treatment of humans.

The invention is described herein in terms of various functional components, compositions and processing steps. It should be appreciated that such components, compositions and steps may be realized by any number of structural components and compositional constituents configured to perform the specified functions. For example, the present invention may employ various compositions and containers for use with systems and kits for reducing tartar; the specific examples as described herein are merely indicative of exemplary applications for the invention. Moreover, the present application refers to numerous aspects and/or embodiments of the invention, some of which are expressly described as being usable in combination with each other or under certain conditions. Any one or more of the features, aspects, and embodiments of the present disclosure may be used in combination with any of the other features, aspects, and/or embodiments. Moreover, references herein to the invention are to be understood broadly as examples of the invention and not as limitations to the claims of the present disclosure.

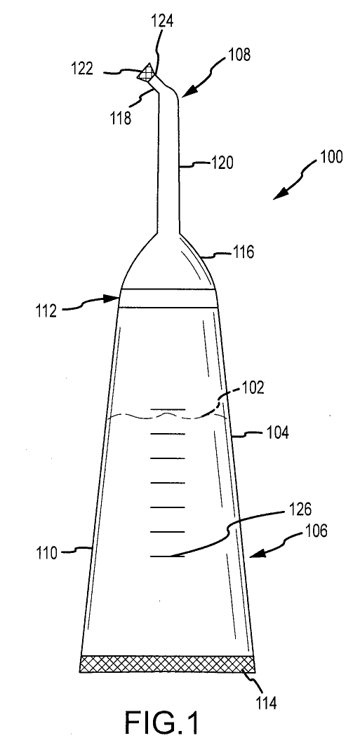

FIG. 1 illustrates a system 100 for use in accordance with various embodiments of the present invention. System 100 is used to facilitate prevention and/or reduction of tartar.

In accordance with various exemplary aspects of the illustrated embodiment, system 100 includes a composition 102 and a container 104 for applying composition 102 to a surface within an oral cavity.

As used throughout this application, the term “surface” includes any surface on which plaque, tartar, or gum disease may form. Exemplary surfaces include teeth (both supragingival and subgingival), gums, and dental devices such as bridges, crowns, fillings, braces, and the like. Further, as used herein, the term “measurably improve” means a measurable difference between an amount measured without use of the composition or system of the present invention and an amount measured with or after use of the system. The measurements may be compared for the same surface (before and after) or between test and control groups.

Container 104 is configured for containment and temporary storage of composition 102, i.e., storage until initiation of the treatment process, and for expulsion and application of composition 102 to a surface to achieve improved oral health and/or hygiene. Container 104 can be configured in various manners for application of composition 102 to a surface. For example, container 104 can comprise various sizes and volumes depending on treatment applications, and/or various shapes and configurations for facilitating delivery of composition 102 to a surface, depending upon, for example, the purpose for which composition 102 is being applied. In the case of prophylaxis or reduction in calculus, it may be desirable to have a multi-dose applicator for convenient, repeated (e.g., daily) application of composition 102. In contrast, a single-dose applicator may be desirable for travel or for applications to specific problem areas, such as targeted application to diseased or infected areas or dental devices within an oral cavity.

In accordance with specific examples of various embodiments, container 104 is configured to store about seven doses, about four doses, about two doses, or about one dose. However, the invention is not necessarily limited to these container sizes.

A dose size may vary in accordance with several factors, such as the particular ingredients, the dilution of the composition, and the like. Exemplary dose sizes for purpose of illustration range from about 1 mg to about 6 mg, preferably about 2 mg to about 5 mg, and more preferably about 3 mg to about 4 mg.

With continued reference to FIG. 1, container 104 includes an expulsion or vessel portion 106 and an applicator portion 108. The vessel portion 106 is configured to contain or store composition 102 and to facilitate expulsion of composition 102. The applicator portion 108 is configured to receive composition 102 from expulsion portion 106 and to facilitate application or delivery of composition 102 to a selected oral cavity surface.

In accordance with particular aspects of this embodiment, vessel portion 106 is formed of a resiliently deformable material that is capable of retaining and returning to its original shape when not under pressure. In accordance with other aspects, vessel portion 106 may be formed of material that does return to its original shape. Exemplary resilient materials suitable for vessel portion 106 include low density polyethylene material, high density polyethylene, medium density polyethylene, linear low density polyethylene, polyvinyl chloride, K resin, polyethylene terephthalate and copolyesters, polypropylene, surlyn, silicones and other thermostatics, metal or alloy, and the like. Portion 106 may be opaque, transparent, or semitransparent. An advantage of forming vessel portion 106 of transparent or semitransparent material is that an amount of material 102 within vessel portion 106 can be ascertained when the portion is formed of such material, which may facilitate dispensing appropriate amounts. Material used to form vessel portion 106 may also include UV protection additives, colorants, or the like, and is preferably FDA-approved material.

In accordance with various embodiments of the invention, expulsion or vessel portion 106 includes a resilient vial 110 and a neck 112. Resilient vial 110 acts as a reservoir for material 102 and also facilitates expulsion of material 102 from system 100 when pressure is applied to an external surface of vial 110. As illustrated, vial 110 may also include graduations 126 to, for example, illustrate a number of doses used and/or a number of doses remaining. Neck 112 is configured to couple to applicator or spout portion 108. A resilient vial 110 and neck 112 are exemplary configurations of the vessel portion 106; other suitable configurations may include a non-resilient cylinder (as an exemplary geometry) with a user operated piston to move along the cylinder to expulse the material.

Although illustrated as substantially tubular, with a sealed end 114, resilient vial 110 may be of any suitable shape. For example, vial 110 may be pyramidal, cone shaped, fluted, or have a rectangular cross section. Similarly, end portion 114 may be of any suitable shape, such as linear (e.g., a crimped or heat-sealed end) or the like. In general, preferred shapes of vial 110 conserve material used to form the vial, allow for easy dispensing of material 102, are easy to produce, and produce relatively little scrap during production.

Container 104 can also be configured to allow a user to suitably control the rate of expulsion into applicator portion 108. For example, in accordance with an exemplary embodiment, container 104 includes a transition region 116 to facilitate flow between vial 110 and spout 108. Transition portion 116 may be of any suitable shape such as frustro-conical, fluted, semi-spherical, and the like. Transition portion 116 may be adapted to restrict or regulate flow by nature of its geometry or may include channels configured to regulate the flow of the composition 102. Additionally or alternatively, container 104 may include one or more features adapted to limit the flow of composition 102 after a predetermined volume of composition has been dispensed. For example, container 104 may include a user-operable lever or other control adapted to dispense a predetermined volume with each push (or other manipulation) of the lever or control.

Applicator portion 108 may be formed of any of the material described above in connection with vessel or expulsion portion 106. Applicator portion 108 is suitably configured for selective or otherwise controlled delivery of composition 102 to a target area within an oral cavity interface. In accordance with various embodiments of the invention, applicator portion 108 is configured to couple (e.g., detachably or otherwise) to expulsion portion 106. Alternatively, applicator portion 108 is configured as a molded or otherwise unitary structure with expulsion portion 106, as described in more detail below. When separately formed, portions 106 and 108 may be coupled using screwed, press-fit, clamped or other techniques to permanently, semi-permanently or removably attached portions 106 and 108. Applicator portion 108 within the scope of the present disclosure may include any suitable features adapted to facilitate or aid in the distribution of the composition onto the user's surface to be treated. For example, applicator portion 108 may include a brush-like portion (not shown) or a spreader portion (not shown) adapted to facilitate the application of the composition to targeted surface areas. Additionally or alternatively, applicator portion 108 may be configured with a directional dispensing tip for dispensing the composition directly on the surface and/or onto auxiliary applicators. Exemplary directional dispensing tips are described in greater detail below.

In some implementations, the system may further include an auxiliary applicator (not shown) that is not adapted to be coupled to vessel portion 106. Exemplary auxiliary applicators may include impression trays or conforming strips that are adapted to receive a quantity of the composition from vessel portion 106 and to apply that composition to user's surface.

In accordance with an exemplary embodiment, applicator portion 108 comprises a structure 120 to allow composition 102 to be forced through an applicator tip 118 and onto a surface within an oral cavity. Structure 120 may be passive and substantially rigid to allow composition to flow from portion 106 to tip 118. Alternatively, structure 120 and/or applicator portion 108 may be configured as less-rigid to allow for expulsion of any remaining composition within applicator portion 108 to be squeezed or otherwise delivered or applied by applicator tip 118 onto a surface.

As illustrated structure 120 may form an angle of about zero degrees with respect to a centerline through expulsion portion 106. Alternatively, structure 120 may form other angles, ranging from about zero degrees to about 90 degrees. In further accordance with the illustrated embodiment, tip 118 forms an angle of about 45 degrees relative to the centerline of component 120; however, tip 118 may suitably form other angles relative to component 120.

Tip 118 is generally configured to facilitate placement and controlled flow of material dispensed from system 100. Tip 118 may be substantially cylindrical. Alternatively, tip 118 may have a square, rectangular, ellipse, or other cross-sectional configuration. Tip 118 may also include a weakened section 124, which may be formed, for example, by scarring a portion of tip 118. Weakened section 124 may facilitate rupturing tip 118 at weakened section 124, which in turn allows for a predicable cross section of tip 118 and thus a relatively predictable flow of material dispensed from system 100. In accordance with one particular example, tip 118 includes a substantially constant cross-section, which makes the flow more predictable, even if tip 118 is ruptured away from weakened area 124.

End portion 122 forms a sealed end at one end of tip 118. In accordance with various embodiments of the invention, end portion 122 is flat and wider at an exterior portion than an interior portion, such that end portion 122 is wider in at least one direction than tip 118. Having end portion 122 wider than tip 118 allows a user to grip end 122 to, for example, tear or sever tip 118 at weakened section 124. However, end 122 may be alternatively configured as, for example, a semisphere or other suitable shape. Alternatively, tip 118 and end portion 122 may be configured, such that end 122 can reattach to tip 118.

Spout portion 108 may be formed using a variety of materials, such as any of the materials described above in connection with vessel portion 106. However, because spout portion 108 may be formed separately from vessel portion 106, it need not be formed of the same material.

In accordance with one embodiment of the invention, vessel portion 106 and spout portion 108 are configured to sealably (and optionally rotatably) couple to each other. In the illustrated embodiment, portion 106 and 108 are threadedly coupled to each other. In accordance with other embodiments, one of portions 106 and 108 includes a protrusion and the other of portion 106, 108 includes a recess to receive the protrusion, such that the protrusion and recess hold vessel portion 106 and spout portion 108 together, while optionally allowing the two portions to rotate about an axis, with respect to each other. In accordance with another embodiment of the invention, portions 106, 108 are configured to allow vessel portion 106 and spout portion 108 to be detachably coupled to each other. In this case, neck 112 and spout 108 may be snap-fit together as described above, or portions 106 and cap 108 may engage using lug or interference-fit technology to sealably attach to each other.

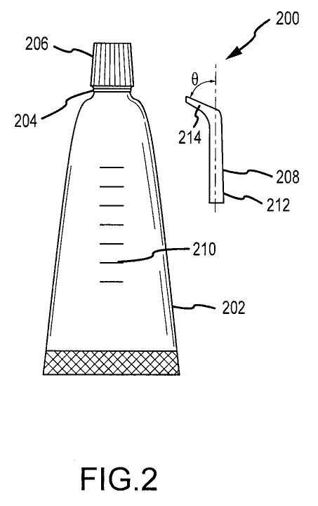

FIG. 2 illustrates another system 200 for use in accordance with additional embodiments of the invention. As illustrated, system 200 includes a vessel portion 202, including a neck 204, a cap 206, and a detachable applicator 208. System 200 is similar to system 100, except system 200 includes resealable cap 206 and detachable applicator 208, rather than applicator portion 108. System 200 may be formed of any of the materials noted above in connection with system 100, and may include graduations 210 to indicated a number of doses used and/or a number of remaining doses, as described above.

Cap 206 can be removably attached to vessel portion 202 using a variety of techniques. For example, cap 206 may be threadedly attached to portion 202. Alternatively, cap 206 and portion 202 may be coupled using snap-fit, lug, interference-fit technology, or similar technologies. In accordance with one specific example of this embodiment, neck 204 includes exterior threads and cap 206 includes interior threads to threadedly engage with neck 204.

Similarly, applicator portion 208 may couple to vessel 202 in a variety of ways, such as threaded, snap-fit, lug, or similar type connections. By way of particular example, applicator 208 threadedly engages with a portion of neck 204.

Applicator 208 includes a first portion 212 and a second or tip portion 214. As illustrated, tip portion 214 is angled relative to a centerline first portion 212; however such is not required for practice of the present invention. Exemplary angles range from about zero to about ninety degrees; one exemplary angle is about forty-five degrees relative to the centerline.

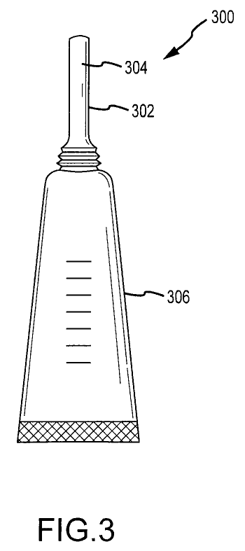

FIG. 3 illustrates yet another system 300 for use in accordance with additional embodiments of the invention. System 300 is similar to system 200, except system 300 includes an applicator 302, rather than applicator 208. Applicator 302 is similar to applicator 208, except applicator 302 does not include an angled tip.

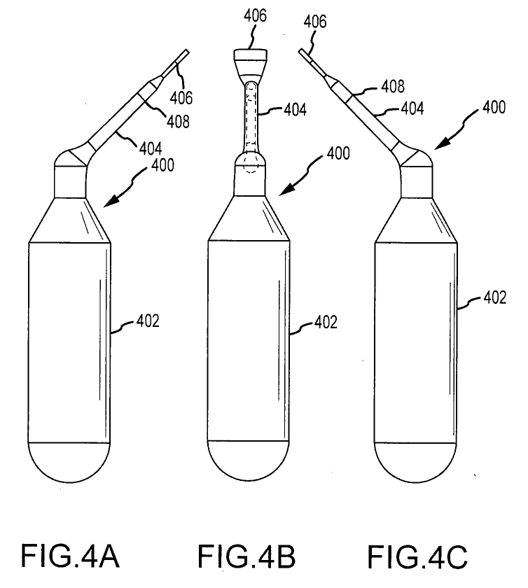

FIGS. 4A-4C illustrate left, front, and right views of yet another system 400 in accordance with various embodiments of the invention. System 400 is similar to systems 100-300, except system 400 is designed as a unitary system, having an integrated vessel 402 and spout 404, including a severable end 406 and scarred section 408.

Although not illustrated, systems for use in accordance with various embodiments of the invention may include tamper-resistant features. For example, system 100 may include a seal formed over neck 112, using, for example plastic or foil glued to or otherwise adhered to a top portion of neck 112. Alternatively, after spout portion 108 is attached to vessel portion 106, the two portions may be fused together using heat sealing and/or adhesive techniques.

In accordance with various exemplary embodiments, a composition (e.g., composition 102), suitable for use with systems (e.g., systems 100, 200, 300) of the present invention comprises an active ingredient and a viscous carrier. In this case, composition 102 is configured to maintain the active ingredient in contact with a surface of an oral cavity for an extended period of time to allow the active ingredient(s) to remain in contact with the surface for an extended period.

Exemplary active ingredients suitable for use with systems of the invention include one or more of the following: cetylpyridinium chloride (CPC), dicalcium phosphate dehydrate, hydrogen peroxide, sanguinaria extract, sodium bicarbonate, sodium lauryl sulfate, sodium fluoride, stannous fluoride, sodium monofluorophosphate (MFP), zinc salts such as zinc chloride, zinc acetate, zinc citrate, zinc oxide and zinc gluconate, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate. Other now known or hereafter devised actives may also be used. For example, any agent, which alone or in combination is able to prevent or alleviate the severity of problems associated with dentition may be utilized. Such may include anti-caries agents and the like; agents useful in reducing tooth hypersensitivity, such as potassium nitrate, strontium chloride and/or the like; and/or plaque and calculus reducing agents, such as, for example, chlorhexidine, quaternary ammonium compounds (e.g. benzethonium chloride, domphen bromide, etc.), triclosan, herbal compounds (e.g. sanguinarine), stannous salts, complex phosphates (e.g., pyrophosphates), SLS (e.g. sodium lauryl sulfate), hydrogen peroxide, and/or the like.

An amount of the active ingredient for use within compositions suitable for uses with the invention varies in accordance with the dosage size and particular ingredient(s). In general, each active or actives selected will be used in a suitably effective amount, generally on the order of less than about 10 wt %, and more preferably 5 wt % or less. An amount of active may also be desirably selected to be within government guidelines, such as guidelines by the Food and Drug Administration in the USA. In particularly preferred compositions, the active ingredient is present in an amount of about 0.001 wt % to about 1.5 wt %, within an amount of about 0.025 wt % to about 1.0 wt %, or even within an amount about 0.05 wt % to about 0.7 wt %. All percentages set forth herein are in weight percent of the total composition, unless otherwise indicated.

In accordance with one preferred exemplary embodiment, the active ingredient(s) include CPC. In one case, CPC is present in an amount of about 0.001% to about 1%, in an amount of about 0.01% to about 0.5%, or even in an amount of about 0.05% to about 0.25% or about 0.045% to about 0.1%. In accordance with another exemplary embodiment, the active ingredient(s) also include zinc gluconate. In one case, zinc gluconate is present in an amount of about 0.001% to about 1.5%, in an amount of about 0.01% to about 1.0%, or even in an amount of about 0.05% to about 0.75%.

In accordance with an exemplary embodiment, composition 102 also includes a thickener to obtain the desired viscosity. Suitable thickening agents include substances which increase the viscosity of composition 102, cause composition 102 to gel or coagulate, or the like, such as food-grade or pharmaceutical-grade thickeners, including, for example, hydroxyethylcellulose, hydroxypropyl methylcellulose, carrageenan, guar gum, methylcellulose, methyethylecellulose, acceptable non-ionic thickeners, and the like. The thickener may be present in an amount of about 0.01% to about 10%, in an amount of about 0.1% to about 7%, or even in an amount of about 1% to about 5% or about 0.5% to about 3%. As described below, the amount of thickener present may be selected based at least in part on the active ingredients and the other components of the composition. For example, the desired viscosity of composition 102 may change based on the quantity and/or effectiveness of the active ingredient(s) or on the salivary response of the user.

Composition 102 may also include a humectant such as glycerin, which may be present in an amount of about 0.01% to about 15%, preferably about 0.1% to about 10%, and more preferably about 1% to about 7%. When used, the humectant may facilitate maintaining composition 102 in a liquid form and may help maintain a desired viscosity. In accordance with specific aspects, glycerin facilitates maintaining one or more of the active ingredients in an ionic form and/or facilitates the transport of the active ingredients through composition 102.

The composition may also include a diluent. Exemplary diluents suitable for use with the present composition include sorbitol, xylitol, mannitol, water, alcohols, and oils. In accordance with particular examples of the invention, the composition includes purified water in an amount of about 80% to about 99%, preferably about 85% to about 95%, and more preferably about 88% to about 92%.

Composition 102 may also include sugar alcohols such as sorbitol and xylitol, monnital, lactitol, and the like that act as a sweetener and also as a humectant and/or emulsifier and/or diluent. When used, sorbitol or other sugar alcohol can be present in an amount of about 0.001% to about 0.5%, in an amount of about 0.01% to about 0.1%, or even in an amount of about 0.025% to about 0.075%.

Composition 102 may also include a natural or artificial sweetener such as cyclamates, sucralose, saccharin (e.g., sodium or calcium), ace-k, or aspartame which, when included in composition 102, can be present in an amount of about 0.001% to about 1.5%, in an amount of about 0.01% to about 1%, or even in an amount of about 0.25% to about 0.75%.

Colorants may also be added to composition 102. For example, composition 102 can include colorants, such that when composition 102 is applied to or proximate the gingiva, composition 102 has a color indicative of healthy gingiva—e.g., composition 102 can be pink in color. Such a composition having a color indicative of healthy gingiva can provide added incentive to users to continue using composition 102, which in turn promotes improved health care and hygiene. Colorants may be present in any desired amount. For example, the colorants may include Red #33 and/or Red #40, available from Pylam in an amount of about 0.000005% to about 1%, preferably about 0.00050% to about 0.5%, and more preferably about 0.001% to about 0.1%. Additionally or alternatively, colorants, which are indicative of flavor may be added to the composition. Examples include FD&C Blue #1, D&C Green #5, FD&C Yellow #5, and FD&C Yellow #6.

Composition 102 may also include flavorants or other additives such as cinnamon oil, clove oil, mints, anise, citrus, fruits, and the like, which, when included in the formula are present in an amount of about 0.01% to about 2%, in an amount of about 0.01% to about 1%, or even in an amount about of about 0.1% to about 0.5%.

Essential oils such as cinnamon bark oil and clove bud oil may be particularly advantageous because they exhibit additional desirable qualities. For example, cinnamon bark oil exhibits antibacterial, antiseptic, antiviral, antispasmodic, antifungal, sedative and analgesic properties and clove bud oil has local anesthetic, antiseptic, antibacterial, and stimulating properties.

Flavorants and sweeteners such as those described above are examples of additives that may be included in the composition to affect the user experience. For example, sweeteners and the flavorants both affect the way the user's taste buds respond to the composition in the mouth. In some implementations, the additives are included merely to modify the taste of the composition. In other implementations, one or more of the additives may have a functional contribution to the effectiveness of the composition. For example, additives may serve as a humectant and/or emulsifier. Moreover, some additives may provide clinical benefits such as described above with the essential oils.

As described below, the viscosity of the composition 102 is selected to provide a composition that will stay in contact with a surface for an extended time. The selection of additives and their concentration in the composition may be informed by the affect such additives will have to the viscosity of the composition and to the affect such additives will have on the user. For example, one or more additives or combinations of additives may be observed to increase the average user salivary response. Depending on the degree of increase, such increased salivary response may result in an otherwise shortened surface contact time of the composition. Accordingly, such additives or combinations of additives may be avoided, their concentration may be reduced to reduce the increased salivary response, or the concentrations of other components may be adjusted to account for the increased salivary response. For example, additional thickener agents may be used to raise the viscosity of the composition when the composition is observed or known to result in increased salivary response.

Composition 102 is configured to maintain contact with an oral surface (or interface) for an extended period of time, which has several advantages over traditional compositions. Composition 102 preferably exhibits good microadhesion, and moreover, composition 102 preferably is quite viscous. As such, in general, relatively small amounts of composition 102 and consequently the active agent(s) can be used to effectively provide oral health care or treatment. Additionally, configuration allows for relatively select placement of the composition on a surface.

Exemplary compositions have a viscosity greater than about 20,000 cp, preferably greater than 30,000 cp, and more preferably greater than about 35,000 cp. By way of more particular examples, the viscosity of compositions range from about 20,000 cp to about 250,000 cp, preferably about 25,000 cp to about 100,000 cp, and more preferably about 30,000 cp to about 50,000 cp, and yet more preferably about 35,000 cp to about 45,000 cp. The viscosity values as set forth herein are measured using a Brookfield, Model DV-II+ Pro viscometer, spindle # 6, 10 RPM for 90 seconds at 25 C.

Within the viscosity ranges described above, the viscosity of the composition may be selected based on any one or more of a variety of factors, such as the cost of the ingredients, the safety of the ingredients and the risks of ingestion, the ease of application, and/or the preferences of the users. Additionally or alternatively, it may be determined that particular viscosities within the ranges described above work better with particular compositions within the scope of the present invention. For example, it may be determined that a less viscous composition is acceptable with certain active ingredients or certain concentrations of active ingredients while a more viscous composition is desired in others. For example, compositions within the scope of the present invention may have a preferred contact time of between 1 hour and 10 hours per night for effective treatment. Compositions with a preferred contact time of just 1 hour may be less viscous than those that have a preferred contact time of 10 hours. The preferred contact time may vary depending on the active ingredient(s) and their concentration(s).

Additionally or alternatively, it may be determined that compositions including certain additives, whether sweeteners, flavorants, or other, are more effective at particular viscosities within the ranges described above. In some methods of using the present compositions, the compositions may be applied to the user's teeth or other dental surfaces at night and allowed to stay in contact with the surface while the user sleeps. Some additives or combinations of additives may cause the user to salivate more (or less) than a user would normally salivate at night. With such additives, it may be preferable to select a higher (or lower) composition viscosity to allow the composition to stay in contact with the surface for the desired amount of time even when the user is salivating more (or less) than normal. In some compositions within the scope of the present invention, the selection and quantity of additives and thickeners may be coordinated to reduce excess salivation by the user and/or to provide the desired viscosity and contact time in light of the expected user response. In some implementations, the compositions may be customized for individual users by adding additional thickeners and/or flavorants if the user has found that the originally provided composition produces unusually high salivation.

In accordance with other exemplary embodiments, composition 102 includes multiple active ingredients in a carrier. For example, composition 102 can include a plurality of any active ingredients and a carrier in the weight percents disclosed herein. Composition 102 may also include any of the optional ingredients, such as thickeners, sweeteners, flavorants, and colorants as set forth herein. For example, in accordance with an exemplary embodiment, composition 102 includes CPC and zinc gluconate as the active ingredients, wherein the CPC is present in an amount of about 0.001% to about 1%, in an amount of about 0.01% to about 0.5%, or even in an amount of about 0.05% to about 0.25% or about 0.045% to about 0.1%; and wherein the zinc gluconate is present in an amount of about 0.001% to about 1.5%, in an amount of about 0.01% to about 1.0%, or even in an amount of about 0.05% to about 0.75%.

In accordance with other exemplary embodiments, composition 102 includes one or more active ingredients and a colorant indicative of healthy gingival tissue. When the colorant is added, the color of composition 102 may be more than merely decorative. For example, the color also serves the function of encouraging those that use the product to continue to use the product because there is an immediate appearance, upon application of composition 102, that healthy gingiva is achieved.

A pH of a composition in accordance with various embodiments of the invention is preferably between about 4-10, more preferably about 4-7, and yet more preferably about 5-5.4.

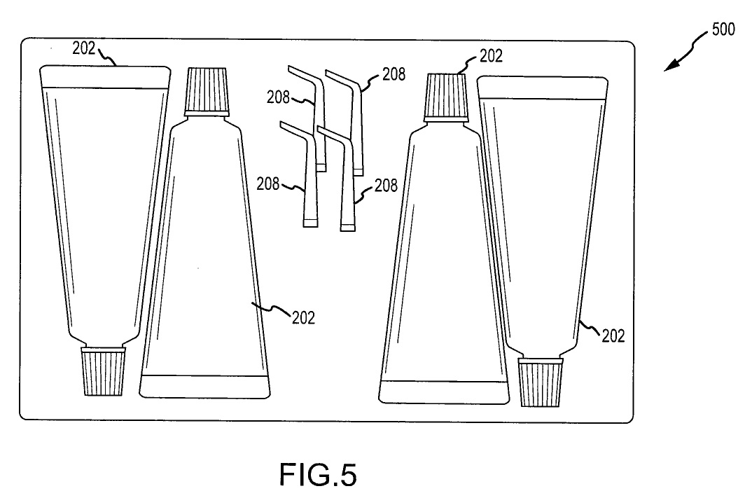

FIG. 5 illustrates a kit 500, including multiple systems, for use in accordance with yet additional embodiments of the invention. As illustrated, kit 500 includes four systems; however, systems in accordance with other embodiments of the invention may include a different number, e.g., 1, 2, 5, 7, 10, or the like number of systems.

In accordance with further exemplary embodiments, with reference to FIG: 6, as well as continuing reference to 1-5, a method 600 of facilitating oral health care includes providing a system (e.g., system 100), including a composition (e.g., composition 102) (step 602) accessing the composition within the container (step 604), and selectively applying the composition to an oral cavity surface (step 606).

Providing a system in accordance with step 602 can include any method now known or hereinafter devised for filling a container with a fluid. With reference to FIG. 1, composition 102 can suitably be filled in one end of container 104 and then sealed to maintain composition 102 within container 104. Alternatively, with reference to FIG. 2, vessel 202 may be filled with composition 102 and then cap 206 sealably attached to vessel 202. In addition, providing composition 102 within container 104 may include providing a single and/or daily dose of composition 102, or multiple doses of composition 102.

Accessing composition 102 within container 104 suitably comprises removal of an access or closure device or component from container 104. For example, in accordance with an exemplary embodiment, accessing composition 102 within container 104 comprises detachably removing an access component, e.g., end portion 122, to provide an access to the composition.

Selectively applying composition 102 to a surface can suitably include expulsing or otherwise forcing or delivering composition 102 from a vial 110 to applicator portion 108 of container 104. For example, in accordance with an exemplary embodiment, composition 102 can be “squeezed” from an expulsion portion 106, into an applicator portion 108, through an applicator tip 118 and onto a targeted region of oral cavity surface. In accordance with various embodiments of the invention, the system does not require any additional additives or solutions for use. That is, the compositions can be applied directly to a surface without diluting or the like. Further, the compositions can be applied without additional devices such as cups or the like. Other exemplary methods of applying the composition to a surface have been described above, including the use of brush-like applicators or other auxiliary applicators, such as impression trays and conforming strips.

Regular and/or systematic use of systems in accordance with various embodiments of the invention onto one or more surfaces results in prevention, mitigation, and/or reduction of tartar on a surface.

A method of forming a composition for use in accordance with various embodiments of the invention includes the steps of adding a humectant (e.g., glycerin) to a first mixing vessel and then adding a thickener (e.g., hydroxyethylcellulose) to the humectant and mixing until a uniform, lump-free slurry forms. The slurry should not sit for too long at this stage, or it may become cement-like in texture and viscosity. In a second mixing vessel, add a diluent (e.g., water) and add the humectant/thickener slurry slowly (over a period of a few hours) to the diluent and mix until a smooth gum mixture is obtained. Once the gum is hydrated, add any sugar alcohol, sweetener, and colorant to the mixture and mix until each is dissolved. Then, add a preservative and mix until dissolved and uniform. Finally, add any oils and mix until the oils are dispersed in the solution. The active ingredient(s) may be added at any time after the smooth gum mixture is obtained. Preferably, the active ingredient is added after the preservative.

### SPECIFIC EXAMPLES

The following non-limiting examples illustrate improvement in oral health using a system, kit, and method in accordance with various embodiments of the invention. These examples are merely illustrative, and it is not intended that the invention be limited to the examples. Systems in accordance with the present invention may include the ingredients listed below as well as additional and/or alternative inert materials, preservatives, and other constituents typically found in compositions for promoting oral health. In the case where exemplary inert materials and/or preservatives are listed, these ingredients are merely exemplary, and it is understood that other similar ingredients may be substituted for the materials listed in the examples below.

### Example 1

A pale light pink viscous gel, having a viscosity of about 40,000 cp, with cinnamon-clove characteristic odor and taste is formed by admixing the following ingredients, as described above, in the amounts shown. The composition was sealed in system 400, illustrated in FIG. 4.

TABLE 1

| | | | Exemplary |
|---|---|---|---|
| Ingredient | Supplier | Weight % | Wt % Range |
| Purified Water | Copacker | 91.504 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Zinc Gluconate | American | 0.592 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

A clinical study, including 24 subjects, evaluating the efficacy of the composition of Table 1 was conducted. Plaque and calculus quality, thickness, maturity, and mass were also observed.

At the end of a three-week period, there was an observed lessening or reduction of plaque quality, thickness, mass, and maturity; the lessening was greater (greater decrease) for those using the composition of Table 1, compared to a placebo. The observance that plaque quality was reduced is important because the presence of actively growing plaque bacteria (biofilm) is important in the development of inflammation, which leads to gingivitis and periodontitis.

In addition, at the end of the three-week period a lessening of the quality of calculus was also observed. A general improvement of gingival health was also observed. An extremely thin, slightly detached layer of epithelial cells was also found on the surface of attached gingival surfaces at the marginal ridge, close to the areas where plaque and tartar was likely disrupted off the teeth with subjects using the composition of Table 1, which indicated promotion of faster healing of gingival tissues.

### Example 2

A clinical study comparing V-MI scores of subjects using the composition of Table 1 was conducted to evaluate calculus dissolution. During the three-month study, the subjects were instructed to brush with toothpaste twice daily and apply the composition prior to retiring. Subjects did not receive professional cleaning just prior to participating in the study. The object of the study was to determine the efficacy of the composition and use thereof at removing calculus.

For 18 subjects using a system including the composition of Table 1, there was a statistically significant difference between original V-MI scores and V-MI scores (51%) after three months of treatment with the product, which indicates the system is effective at calculus dissolution.

### Example 3

In another clinical study, 50 subjects received professional prophylaxis, including scaling and polishing, to remove supragingival calculus, extrinsic stain, and plaque deposits from the mandibular anterior teeth and initial V-MI measurements at 18 VM sites per subject were recorded prior to the study to evaluate calculus inhibition. Subjects were requested to refrain from flossing the mandibular 6 anterior teeth and place the composition of Table 1 between and around those teeth using system 400, illustrated in FIG. 4. At the end of a three-month period, VM-I measurements for the same teeth were recorded and analyzed using analysis of covariance. The V-MI scores at the end of the three-month period were statistically significantly lower (59%) than the initial V-MI scores. The study indicated that the system was effective at Calculus inhibition.

### Example 4

A clinical study comparing V-MI scores of 40 subjects using the composition of Table 1 was conducted to evaluate disruption or dissolution of existing calculus bridges. During a three-month study, the subjects were instructed to brush with toothpaste twice daily and apply the composition prior to retiring.

At the end of the three-month study, a statistically significant difference (30% difference) of before and after scores of the subjects using the composition of Table 1 was observed. In addition, a statistically significant difference (26% difference) of the 40 subjects compared to 40 subjects using a placebo was observed, and a statistically significant difference (99%) between the change in scores before and after the study between the placebo group and the group using a system including the composition of Table 1.

### Example 5

A clinical study comparing VMI scores using a placebo and the compositions of Tables 1 and 4 was conducted. In this study, 90 subjects were enrolled and 84 completed. Data was recorded at baseline, 40 days and 90 days. Subjects were provided coded tubes of the gel formulation to which they were assigned. The subjects were instructed to apply the gel daily; once before going to bed. The gel was applied between the teeth at the gum margin all along the arch. After applying, the subjects were asked to spit out any excess but not to eat or drink.

TABLE 4

| | | | Exemplary |
|---|---|---|---|
| Ingredient | Supplier | Weight % | Wt % Range |
| Purified Water | Copacker | 91.504 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Zinc Gluconate | American | 1.184 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

The composition of Table 4 appeared to be superior to the composition of Table 1 in terms of percent change in VMI. For example, after 90 days, users of the composition of Table 4 had 19% better VMI scores, while users of the composition of Table 1 had 10% better VMI scores. Moreover, scoring of lingual calculus reduction from intraoral images suggested that the compositions of Tables 1 and 4 were more effective than the placebo. Following treatment, users of the composition of Table 1 had 0.31 better absolute VMI scores and users of the composition of Table 4 had 0.32 better absolute VMI scores, while users of the placebo only had 0.17 better absolute VMI scores, suggesting that the compositions of Tables 1 and 4 both were more effective at calculus reduction than the placebo.

The present invention has been described above with reference to various exemplary embodiments. However, those skilled in the art will recognize that changes and modifications may be made to the exemplary embodiments without departing from the scope of the present invention. For example, the various operational steps, as well as the components for carrying out the operational steps, may be implemented in alternate ways depending upon the particular application or in consideration of any number of cost functions associated with the operation of the system, e.g., various of the steps may be deleted, modified, or combined with other steps. These and other changes or modifications are intended to be included within the scope of the present invention, as set forth in the following claims.

1. A method of reducing tartar on a surface, the method comprising:
providing a system comprising a container and a composition, wherein the composition includes at least one active ingredient for reducing dental deposits and has a viscosity greater than about 20,000 cp, wherein the container includes a vessel portion adapted to store the composition and an applicator portion adapted to cooperate with the vessel portion to communicate at least some of the composition to a surface;
discharging the composition from the vessel portion to the applicator portion;
moving the applicator portion over a surface to apply the composition to the surface; and
allowing the composition to remain in contact with the surface for an extended time.

2. The method of claim 1, wherein the vessel portion and the applicator portion form an integral container, and wherein the container is configured to apply the composition directly to the surface.

3. The method of claim 1, wherein moving the applicator portion over the surface applies the composition and spreads the composition over the surface.

4. The method of claim 1, wherein the applicator portion is separate from the vessel portion.

5. The method of claim 1, wherein the applicator portion is selected from the group consisting of a brush-like applicator and a directional dispensing tip.

6. The method of claim 1, wherein the system further comprises an auxiliary applicator adapted to be placed in contact with the surface and left in place for an extended time.

7. The method of claim 1, wherein the discharging step includes discharging a single dose of the composition to the applicator portion.

8. The method of claim 1, wherein the container includes indicia of dosing levels, and wherein discharging the composition from the vessel portion to the applicator portion stops about when the indicia on the container indicates that a single dose has been discharged.

9. The method of claim 8, wherein the container is configured to stop discharging the composition after discharging a single dose.

10. The method of claim 1, wherein the at least one active ingredient is selected from the group consisting of cetylpyridinium chloride, dicalcium phosphate dehydrate, hydrogen peroxide, sanguinaria extract, sodium bicarbonate, sodium lauryl sulfate, stannous fluoride, zinc salts, alkyl dimethyl amine oxide, akyl dimethyl glycine, eucalyptol, menthol, methyl salicylate, thymol, sodium citrate, peppermint oil, sage oil, polymethylsiloxane, poloxamer, and pyrophosphates.

11. The method of claim 10, wherein the at least one active ingredient includes an active ingredient selected from the group consisting of cetylpyridinium chloride and zinc gluconate.

12. The method of claim 11, wherein the at least one active ingredient includes at least about 0.10 wt % cetylpyridinium chloride and at least about 0.592 wt. % zinc gluconate.

13. The method of claim 1, wherein the composition includes at least one thickener selected to provide a composition having a surface contact time of greater than about four hours.

14. A method of reducing tartar on a surface, the method comprising:
providing a composition including a plurality of active ingredients, at least one thickener ingredient, and at least one additive ingredient in a carrier adapted to have an extended surface contact time;
applying the composition to a dental surface; and
allowing the composition to remain in contact with the surface for an extended time.

15. The method of claim 14, wherein the composition has a viscosity of greater than about 20,000 cp.

16. The method of claim 14, wherein the plurality of active ingredients are selected from the group consisting of cetylpyridinium chloride, dicalcium phosphate dehydrate, hydrogen peroxide, sanguinaria extract, sodium bicarbonate, sodium lauryl sulfate, stannous fluoride, zinc salts, alkyl dimethyl amine oxide, akyl dimethyl glycine, eucalyptol, menthol, methyl salicylate, thymol, sodium citrate, peppermint oil, sage oil, polymethylsiloxane, poloxamer, and pyrophosphates.

17. The method of claim 16, wherein the plurality of active ingredients includes cetylpyridinium chloride and zinc gluconate.

18. The method of claim 17, wherein the at least one active ingredient includes at least about 0.10 wt % cetylpyridinium chloride and at least about 0.592 wt. % zinc gluconate.

19. The method of claim 14, wherein the at least one additive is selected from the group consisting of sweeteners and flavorants.

20. The method of claim 14, wherein the concentration of the at least one thickener ingredient is increased when the at least one additive ingredient or the at least one active ingredient is known to increase a user's salivary response.

21. A method of reducing tartar on a surface, the method comprising:
providing a composition including cetylpyridinium chloride, a zinc salt, and a carrier;
cleaning a user's dental surface;
applying the composition to the dental surface prior to the user resting; and
allowing the composition to remain in contact with the dental surface for an extended time.

22. The method of claim 21, wherein the composition includes between about 0.001 wt % and about 1.0 wt % cetylpyridinium chloride and between about 0.001 wt % and about 1.5 wt % zinc gluconate.

23. The method of claim 21, wherein the composition includes between about 0.05 wt % and about 0.25 wt % cetylpyridinium chloride and between about 0.05 wt % and about 0.75 wt % zinc gluconate.
